(19) United States
(12) Patent Application Publication
(10) Pub. No.: US 2026/0321370 A1
(43) Pub. Date: Oct. 15, 2026

(54) HEALTH MANAGEMENT SYSTEM

(71) Applicant: Japan Display Inc., Tokyo (JP)

(72) Inventors: Kotaro TANAKA, Tokyo (JP); Takenori HIROTA, Tokyo (JP); Yukio TAKAYASHIKI, Tokyo (JP); Masashi TAKAHATA, Tokyo (JP)

(21) Appl. No.: 19/696,635

(22) Filed: Jun. 3, 2026

Related U.S. Application Data
(63) Continuation of application No. PCT/JP2024/043817, filed on Dec. 11, 2024.

(30) Foreign Application Priority Data
Dec. 13, 2023 (JP) 2023-210443

(51) Int. Cl. G16H 10/60; A61B 5/00

(52) U.S. Cl. CPC G16H 10/60; A61B 5/0059; A61B 5/6802; A61B 5/742

(57) ABSTRACT

According to an aspect, a health management system includes: a server; a user terminal device through which a user in a customer uploads biometric information to the server; an operator-side monitoring management terminal device through which the biometric information uploaded to the server is viewable and monitoring information on health management for the user is addable to the biometric information; and a customer-side monitoring management terminal device through which the biometric information uploaded to the server and the monitoring information added to the biometric information are viewable.

### CROSS-REFERENCE TO RELATED APPLICATION

This application claims the benefit of priority from Japanese Patent Application No. 2023-210443 filed on Dec. 13, 2023 and International Patent Application No. PCT/JP2024/043817 filed on Dec. 11, 2024, the entire contents of which are incorporated herein by reference.

### BACKGROUND

### 1. Technical Field

What is disclosed herein relates to a health management system.

### 2. Description of the Related Art

It is desired to acquire biometric information and activity logs of users of biometric sensors, and effectively use such information and logs to manage health of the users and reduce psychological loads on the users. Examples of the biometric information include pulse waves, heart rates, and body temperature. Examples of the activity logs include step counts. Examples of the biometric sensors include smart rings. For example, Japanese Patent Application Laid-open Publication No. 2023-061879 discloses a biometric information monitoring system. In the biometric information monitoring system, physical activity data of a subject acquired by sensors on a client side is transmitted to a cloud server; and the biometric information acquired by the cloud server is analyzed using a predetermined analysis method, and the analysis results are kept on the cloud server. Then, on demand from the subject, the analysis results of the biometric information of the subject are fed back from the cloud server and received by the subject.

In the conventional technology described above, the analysis results on each subject are viewable by the subject, and in order for a third person to evaluate the analysis results, the subject needs to disclose the analysis results to the third person. For this reason, when, for example, an abnormality occurs in health conditions of the subject, a response thereto may be delayed.

There is a desire to build a health management system that efficiently manages the health of members of customers, such as corporations, public institutions, and the like, on a customer-by-customer basis.

For the foregoing reasons, there is a need for a health management system that can visualize health conditions of a user and achieve efficient health management.

### SUMMARY

According to an aspect, a health management system includes: a server; a user terminal device through which a user in a customer uploads biometric information to the server; an operator-side monitoring management terminal device through which the biometric information uploaded to the server is viewable and monitoring information on health management for the user is addable to the biometric information; and a customer-side monitoring management terminal device through which the biometric information uploaded to the server and the monitoring information added to the biometric information are viewable.

### BRIEF DESCRIPTION OF THE DRAWINGS

FIG. 1 is a schematic diagram illustrating a specific example of a business model to which a health management system according to an embodiment is applied;

FIG. 2 is a schematic diagram illustrating an example of a biometric information acquisition device;

FIG. 3 is a schematic sectional view taken along II-II′ illustrated in FIG. 2;

FIG. 4 is a schematic partial sectional view of each of a first optical sensor and a second optical sensor;

FIG. 5 is a conceptual diagram illustrating an example of databases provided on a cloud server;

FIG. 6 is a conceptual diagram illustrating an example of a procedure for managing a master database (DB);

FIG. 7 is a conceptual diagram illustrating an example of a procedure for managing a user information table;

FIG. 8 is a conceptual diagram illustrating an example of a procedure for updating a biometric information table;

FIG. 9 is a conceptual diagram illustrating an example of a procedure for managing the biometric information table;

FIG. 10 is a conceptual diagram illustrating an example of a procedure for transmitting a user abnormality notification; and

FIG. 11 is a flowchart illustrating an example of a process for determination of a biometric information abnormality by the cloud server.

### DETAILED DESCRIPTION

The following describes a mode (embodiment) for carrying out the present disclosure in detail with reference to the drawings. The present disclosure is not limited to the description of the embodiment given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the present disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same element as that described with reference to an already mentioned drawing is denoted by the same reference numeral through the present specification and the drawings, and detailed description thereof may not be repeated where appropriate.

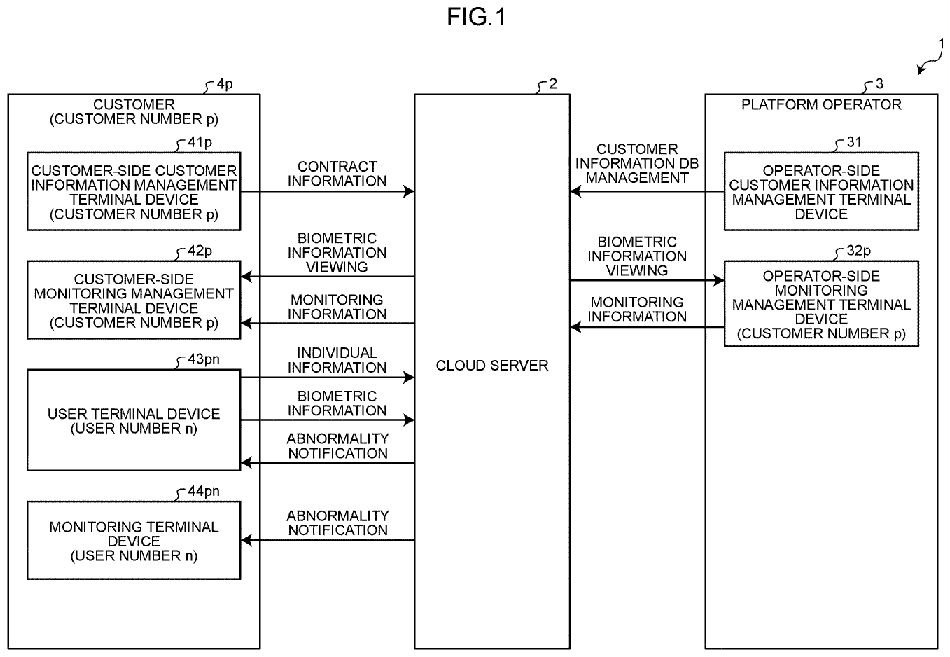

FIG. 1 is a schematic diagram illustrating a specific example of a business model to which a health management system according to the embodiment is applied. The business model illustrated in FIG. 1 includes a cloud server (server) 2, a platform operator 3, and a customer 4p.

The platform operator 3 develops and operates a platform used to implement the business model illustrated in FIG. 1. The platform is a business-to-business (B-to-B) platform built on a cloud network. The platform operator 3 performs health management for a user of a biometric information acquisition device (hereinafter, also simply called “user”) for the customer 4p, such as a corporation or a public institution. Examples of the biometric information acquisition device owned by the user include, but are not limited to, what is called a smart ring.

In the present disclosure, p indicates a customer number of a customer 4p among the total number P (where P is a natural number of 1 or greater) of the customers 4p. For ease of explanation, an aspect is exemplified where a unique customer number p is assigned to each customer 4p, but a unique customer identifier (ID) corresponding to each customer 4p may be assigned instead of the customer number p.

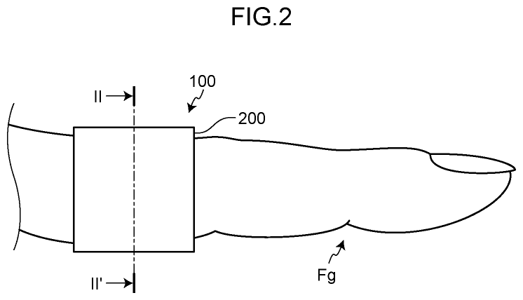

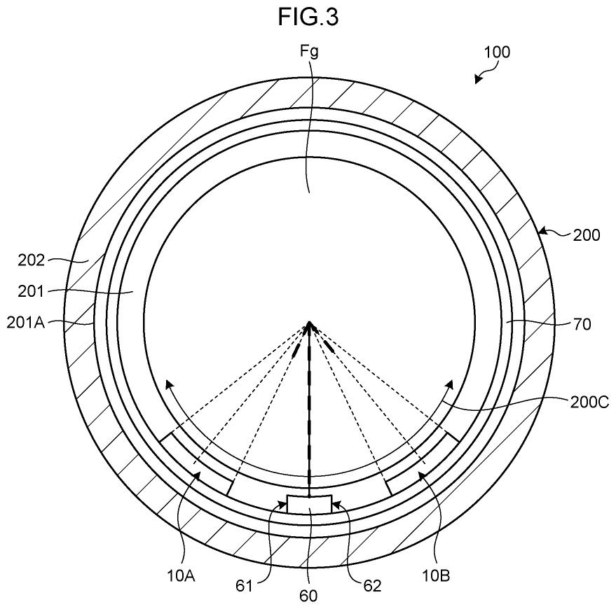

FIG. 2 is a schematic diagram illustrating the biometric information acquisition device. FIG. 3 is a schematic sectional view taken along II-II′ illustrated in FIG. 2. Examples of a biometric information acquisition device 100 include, but are not limited to, a smart ring in an aspect illustrated in FIGS. 2 and 3.

The biometric information acquisition device 100 illustrated in FIGS. 2 and 3 is a finger ring-shaped device that can be worn on and removed from a human body and is worn on a finger Fg of the human body. Examples of the finger Fg include a thumb, an index finger, a middle finger, a ring finger, and a little finger. The biometric information acquisition device 100 can detect biometric information of a living body from the finger Fg wearing the biometric information acquisition device 100. The biometric information acquisition device 100 is not limited to the finger ring-shaped device in the aspect illustrated in FIGS. 2 and 3. The biometric information acquisition device 100 can have any of various shapes, such as that of an arm ring-shaped device worn on an arm of a human body or a device that can be attached to a surface of a skin. In the following description, the biometric information acquisition device 100 is assumed to be used as a finger ring.

As illustrated in FIG. 3, the biometric information acquisition device 100 includes a housing 200, a light source 60, a first optical sensor 10A, and a second optical sensor 10B. The biometric information acquisition device 100 is a device that includes a battery (not illustrated) in the housing 200 and is operated by power of the battery.

The housing 200 is a wearable member that is formed in a ring shape (annular shape) wearable on the finger Fg, and is worn on the living body.

In the example illustrated in FIG. 3, the housing 200 includes a sealing film 201 and an exterior 202. The housing 200 is formed in the ring shape in such a manner that the sealing film 201 is integrated with the exterior 202.

The sealing film 201 accommodates therein the light source 60, the first optical sensor 10A, the second optical sensor 10B, and other components. The sealing film 201 is formed in a ring shape using a housing material, such as a light-transmitting synthetic resin or silicone.

The exterior 202 has a surface of the housing 200 that covers an outer peripheral surface 201A of the sealing film 201. The exterior 202 is formed in a ring shape using a member of, for example, a metal, a non-light-transmitting synthetic resin, or the like.

The sealing film 201 of the housing 200 accommodates a flexible printed circuit board 70 on which the light source 60, the first optical sensor 10A, the second optical sensor 10B, and other components are formed. The flexible printed circuit board 70 is accommodated in the housing 200 by, for example, being formed into a ring shape and, in that state, filling a periphery thereof with a filling member in a mold to form the housing 200.

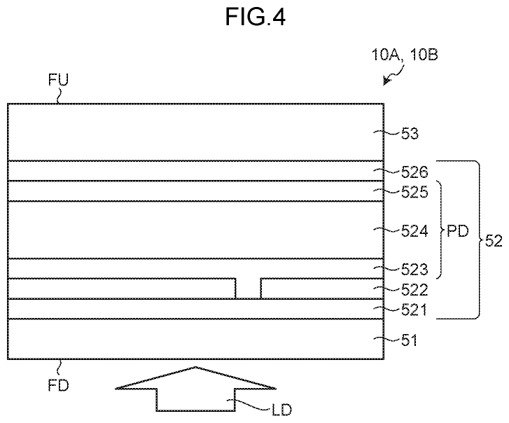

FIG. 4 is a schematic partial sectional view of each of the first optical sensor and the second optical sensor. Each of the first optical sensor 10A and the second optical sensor 10B includes a sensor base member 51, a sensor structure 52, and a protective film 53. The sensor base member 51 is, for example, an insulating base member formed of a film-like resin. An optical sensor PD is an organic photodiode (OPD), for example, and outputs an electrical signal corresponding to light emitted thereto as a detection signal.

The sensor structure 52 includes a thin-film transistor (TFT) layer 521, an anode electrode (lower electrode) 522, the optical sensor PD, and a cathode electrode (upper electrode) 526.

The TFT layer 521 is provided with various types of wiring such as a gate line GCL and a signal line SGL. The sensor base member 51 and the TFT layer 521 are a drive circuit that drives the sensor, and are also called a backplane.

The optical sensor PD includes an active layer 524, an electron transport layer (lower buffer layer) 523 provided between the active layer 524 and the anode electrode (lower electrode) 522, and a hole transport layer (upper buffer layer) 525 provided between the active layer 524 and the cathode electrode (upper electrode) 526. In this case, the electron transport layer (lower buffer layer) 523, the active layer 524, and the hole transport layer (upper buffer layer) 525 of the optical sensor PD are stacked in this order in a direction perpendicular to the sensor base member 51.

The active layer 524 changes in characteristics (for example, voltage-current characteristics and resistance value) depending on light emitted thereto. An organic material is used as a material of the active layer 524. Specifically, the active layer 524 has a bulk heterostructure containing a mixture of a p-type organic semiconductor and an n-type fullerene derivative ((6,6)-phenyl-C₆₁-butyric acid methyl ester (PCBM)) that is an n-type organic semiconductor. As the active layer 524, low-molecular-weight organic materials can be used including, for example, fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The active layer 524 can be formed by a vapor deposition process (dry process) using any of the low-molecular-weight organic materials listed above. In this case, the active layer 524 may be, for example, a multilayered film of CuPc and F₁₆CuPc, or a multilayered film of rubrene and C₆₀.

The active layer 524 can also be formed by a coating process (wet process). In this case, the active layer 524 is made using a material obtained by combining any of the above-listed low-molecular-weight organic materials with a high-molecular-weight organic material.

As the high-molecular-weight organic material, for example, poly(3-hexylthiophene) (P3HT) and F8-alt-benzothiadiazole (F8BT) can be used. The active layer 524 can be a film made of a mixture of P3HT and PCBM, or a film made of a mixture of F8BT and PDI.

The electron transport layer (lower buffer layer) 523 and the hole transport layer (upper buffer layer) 525 are provided to facilitate the transfer of electrons and holes, which have been generated in the active layer 524, to the anode electrode (lower electrode) 522 or the cathode electrode (upper electrode) 526.

The electron transport layer (lower buffer layer) 523 is in direct contact with the top of the anode electrode (lower electrode) 522. The active layer 524 is in direct contact with the top of the electron transport layer (lower buffer layer) 523. Polyethylenimine ethoxylated (PEIE) is used as a material of the electron transport layer (lower buffer layer) 523.

The hole transport layer (upper buffer layer) 525 is in direct contact with the top of the active layer 524, and the cathode electrode (upper electrode) 526 is in direct contact with the top of the hole transport layer (upper buffer layer) 525. The hole transport layer (upper buffer layer) 525 is a metal oxide layer. Tungsten oxide (WO₃), molybdenum oxide, or the like is used as the metal oxide layer.

The materials and the manufacturing methods of the electron transport layer (lower buffer layer) 523, the active layer 524, and the hole transport layer (upper buffer layer) 525 are merely exemplary, and other materials and manufacturing methods may be used.

The anode electrode (lower electrode) 522 faces the cathode electrode (upper electrode) 526 with the optical sensor PD interposed therebetween. A light-transmitting conductive material such as indium tin oxide (ITO) is used as the cathode electrode (upper electrode) 526. A metal material such as silver (Ag) or aluminum (Al) is used as the anode electrode (lower electrode) 522. Alternatively, the anode electrode (lower electrode) 522 may be an alloy material containing at least one or more of these metal materials.

The anode electrode (lower electrode) 522 can be formed as a light-transmitting transflective electrode by controlling the film thickness of the anode electrode (lower electrode) 522. For example, the anode electrode (lower electrode) 522 is formed of an Ag thin film having a thickness of 10 nm so as to have light transmittance of approximately 60%. In this case, the optical sensor PD can detect, for example, first light LD emitted to a first surface FD side.

The protective film 53 is provided on a second surface FU so as to cover the cathode electrode (upper electrode) 526. The protective film 53 is a passivation film and is provided to protect the optical sensor PD.

Unlike in the configuration described above, the optical sensor PD may have a configuration with the active layer 524, a hole transport layer (lower buffer layer) 523 provided between the active layer 524 and the cathode electrode (lower electrode) 522, and an electron transport layer (upper buffer layer) 525 provided between the active layer 524 and the anode electrode (upper electrode) 526. In this case, the hole transport layer (lower buffer layer) 523, the active layer 524, and the electron transport layer (upper buffer layer) 525 of the optical sensor PD are stacked in this order in the direction perpendicular to the sensor base member 51.

In the present disclosure, the optical sensor PD is not limited to an organic photodiode (OPD). The optical sensor PD may be a silicon photodiode (SiPD), for example.

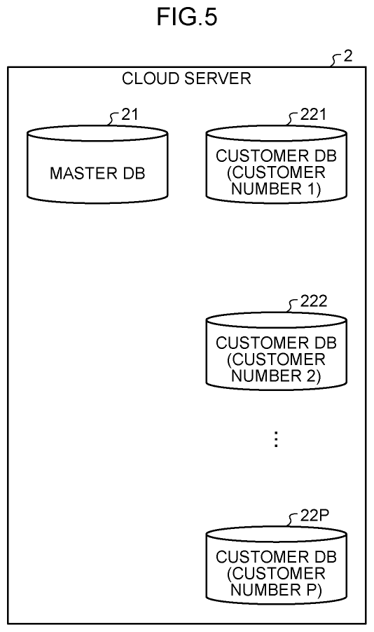

FIG. 5 is a conceptual diagram illustrating an example of databases provided on the cloud server. The cloud server 2 includes a master database (hereinafter, referred to as “master DB”) 21, and customer databases (hereinafter, referred to as “customer DBs”) 221, 222, . . . , 22P. The customer DBs 22p (221, 222, . . . , 22P) are provided, one for each of the customers 4p (41, 42, . . . , 4P).

The master DB 21 stores therein contract information in the business model illustrated in FIG. 1. The contract information for each of the customers 4p is associated with the customer number (customer ID).

The customer DBs 22p are provided corresponding to the customers 4p. Each of the customer DBs 22p stores therein a user information table and a biometric information table. Each of the customer DBs 22p is associated with the corresponding customer number (customer ID).

The user information table stores therein individual information for each user of each customer 4p. In the present disclosure, the user information table stores, in association with one another, at least the following data items as the user's individual information: a user number (user ID) of each user; contact information for a monitoring person (hereinafter, referred to as a monitor) designated by the user; and other information. Examples of the contact information for the monitor include, but are not limited to, an electronic mail address via which a monitor terminal device (monitoring terminal device 44p) to be described later can receive messages. Examples of the monitor designated by the user include, but are not limited to, close relatives of the user. More than one monitor may be designated by the user.

The biometric information table stores therein the biometric information of each user of each customer 4p. In the present disclosure, examples of the biometric information of each user stored in the biometric information table include, but are not limited to, pulse waves, heart rates, body temperature, blood oxygen saturation levels (SpO₂, which may be commonly referred to as “blood oxygen wellness”). As an aspect, the biometric information may include, for example, information about activity, such as walking and/or running, of the user.

A health management system 1 according to the embodiment includes, as operator-side terminal devices, an operator-side customer information management terminal device 31 and an operator-side monitoring management terminal device 32p.

The operator-side customer information management terminal device 31 and the operator-side monitoring management terminal device 32p are terminal devices owned by the platform operator 3. The operator-side monitoring management terminal device 32p is provided for each of the customers 4p.

The operator-side customer information management terminal device 31 and the operator-side monitoring management terminal device 32p are, for example, personal computers (hereinafter, also called “PCs”), but are not limited to the PCs. The operator-side customer information management terminal device 31 may be, for example, a tablet computer or a smartphone owned by an operator-side customer information management staff. The operator-side monitoring management terminal device 32p may be, for example, a tablet computer or a smartphone owned by an operator-side monitoring management staff.

The operator-side customer information management terminal device 31 is not limited to a specific terminal device. The operator-side customer information management terminal device 31 only needs to be capable of executing a password-protected operator-side customer information management application.

The operator-side monitoring management terminal device 32p is not limited to a specific terminal device. The operator-side monitoring management terminal device 32p only needs to be capable of executing a password-protected operator-side monitoring management application.

The operator-side customer information management staff uses the operator-side customer information management terminal device 31 to manage the master DB 21 on the cloud server 2.

The operator-side monitoring management staff has authority to view the biometric information table of each of the customers 4p assigned to the staff. In the present disclosure, the operator-side monitoring management staff logs into the password-protected operator-side monitoring management application to view the biometric information table of each of the customers 4p assigned to the staff. In other words, each of the multiple operator-side monitoring management terminal devices 32p corresponding to the customers 4p has authority to access the customer DB 22p storing therein the biometric information table of the corresponding customer 4p.

Specifically, the operator-side monitoring management terminal device 32p accesses the customer DB 22p. The cloud server 2 determines whether the operator-side monitoring management terminal device 32p has authority to access the customer DB 22p based on the contract information associated with the customer number (customer ID). If the operator-side monitoring management terminal device 32p has authority to access the customer DB 22p, the cloud server 2 allows the operator-side monitoring management terminal device 32p to access the customer DB 22p. Thus, the security among the customers 4p is ensured.

By using the operator-side monitoring management terminal device 32p, the operator-side monitoring management staff can view the biometric information table of the customer 4p and add thereto information on the health management for the user as monitoring information. Thus, the qualitative improvement in the health management for the user can be expected.

The health management system 1 according to the embodiment also includes, as customer-side terminal devices, a customer-side customer information management terminal device 41p, a customer-side monitoring management terminal device 42p, a user terminal device 43pn, and a monitoring terminal device 44pn.

In the present disclosure, n denotes the user number of the user of the biometric information acquisition device among a total number N of users (where N is a natural number of 1 or greater) in the customer 4p. For ease of explanation, an aspect is exemplified where a unique user number n is assigned to each user of the biometric information acquisition device in the customer 4p, but a unique user ID corresponding to each user of the biometric information acquisition device in the customer 4p may be assigned instead of the user number n.

The customer-side customer information management terminal device 41p and the customer-side monitoring management terminal device 42p are, for example, PCs, but are not limited to PCs. The customer-side customer information management terminal device 41p may be, for example, a tablet computer or a smartphone owned by a customer-side customer information management staff of the customer 4p. The customer-side monitoring management terminal device 42p may be, for example, a tablet computer or a smartphone owned by a customer-side monitoring management staff.

The customer-side customer information management terminal device 41p is not limited to a specific terminal device. The customer-side customer information management terminal device 41p only needs to be capable of executing a password-protected customer-side customer information management application.

The customer-side monitoring management terminal device 42p is not limited to a specific terminal device. The customer-side monitoring management terminal device 42p only needs to be capable of executing a password-protected customer-side monitoring management application.

The customer-side customer information management staff uses the customer-side customer information management terminal device 41p to register and update customer information for the customer 4p.

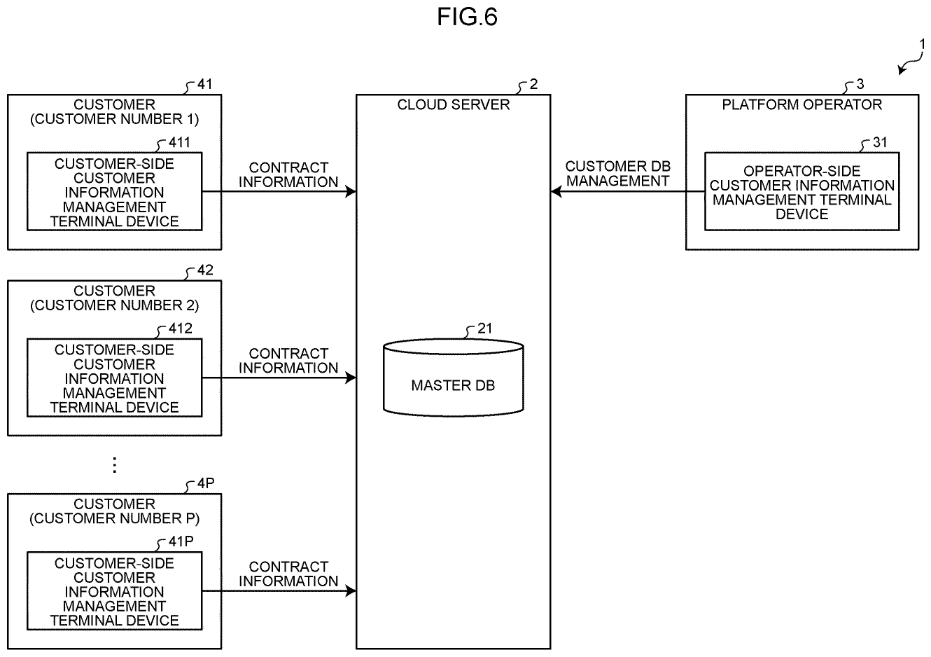

FIG. 6 is a conceptual diagram illustrating an example of a procedure for managing the master DB. In the present disclosure, the customer-side customer information management staff of each of the customers 4p logs into the password-protected customer-side customer information management application to register and update the customer information for the customer 4p. Thus, the security among the customers 4p is ensured.

Specifically, the contract information for the customer 41 transmitted from a customer-side customer information management terminal device 411 of the customer 41 is stored in the master DB 21 on the cloud server 2.

The contract information for the customer 42 transmitted from a customer-side customer information management terminal device 412 of the customer 42 is stored in the master DB 21 on the cloud server 2.

The contract information for the customer 4P transmitted from a customer-side customer information management terminal device 41P of the customer 4P is stored in the master DB 21 on the cloud server 2.

The operator-side customer information management staff of the platform operator 3 uses the operator-side customer information management terminal device 31 to manage the contract information for each customer 4p stored in the master DB 21 on the cloud server 2.

The customer-side monitoring management staff has authority to view the biometric information table of the customer 4p. In the present disclosure, the customer-side monitoring management staff logs into the password-protected customer-side monitoring management application to view the biometric information table of the customer 4p. In other words, the customer-side monitoring management terminal device 42p owned by each of the customers 4p has authority to access the customer DB 22p storing therein the biometric information table of the customer 4p owning the customer-side monitoring management terminal device 42p.

Specifically, the customer-side monitoring management terminal device 42p accesses the customer DB 22p. The cloud server 2 determines whether the customer-side monitoring management terminal device 42p has authority to access the customer DB 22p based on the contract information associated with the customer number (customer ID). If the customer-side monitoring management terminal device 42p has authority to access the customer DB 22p, the cloud server 2 allows the customer-side monitoring management terminal device 42p to access the customer DB 22p. Thus, the security among the customers 4p is ensured.

By using the customer-side monitoring management terminal device 42p, the customer-side monitoring management staff can view the biometric information table of the customer 4p and refer to the monitoring information added by the operator-side monitoring management staff as the information on the health management for the users. Thus, the qualitative improvement in the health management for the users can be expected.

The user terminal device 43pn is, for example, a smartphone owned by the user, but is not limited to the smartphone. The user terminal device 43pn may include, for example, a biometric information acquisition device, such as a smart ring.

The user terminal device 43pn has a function to transmit the individual information and the biometric information of the user to the cloud server 2. The individual information and the biometric information of the user transmitted from the user terminal device 43pn are stored in the customer DB 22p on the cloud server 2.

The user terminal device 43pn is not limited to a specific terminal device. The user terminal device 43pn only needs to be capable of executing a password-protected individual information management application and a password-protected biometric information acquisition application.

The monitoring terminal device 44pn is, for example, a smartphone owned by the monitor designated by the user, but is not limited to the smartphone. The monitoring terminal device 44pn may be, for example, a PC or a tablet computer.

The monitoring terminal device 44pn has a function to receive a user abnormality notification transmitted from the cloud server 2. If the cloud server 2 detects an occurrence of an abnormality in health conditions of the user, the user abnormality notification is transmitted to both the user terminal device 43pn and the monitoring terminal device 44pn.

The monitoring terminal device 44pn is not limited to a specific terminal device. The monitoring terminal device 44pn only needs to be capable of receiving the user abnormality notification transmitted from the cloud server 2. For example, the user abnormality notification is transmitted from the cloud server 2 to the monitoring terminal device 44pn via electronic mail. The transmission of the user abnormality notification is not limited thereto. The user abnormality notification may be transmitted from the cloud server 2 to the monitoring terminal device 44pn via a dedicated application operating on the monitoring terminal device 44pn.

The user abnormality notification may be transmitted from the cloud server 2 to the user terminal device 43pn via the biometric information acquisition application, or via electronic mail in the same way as the monitoring terminal device 44pn.

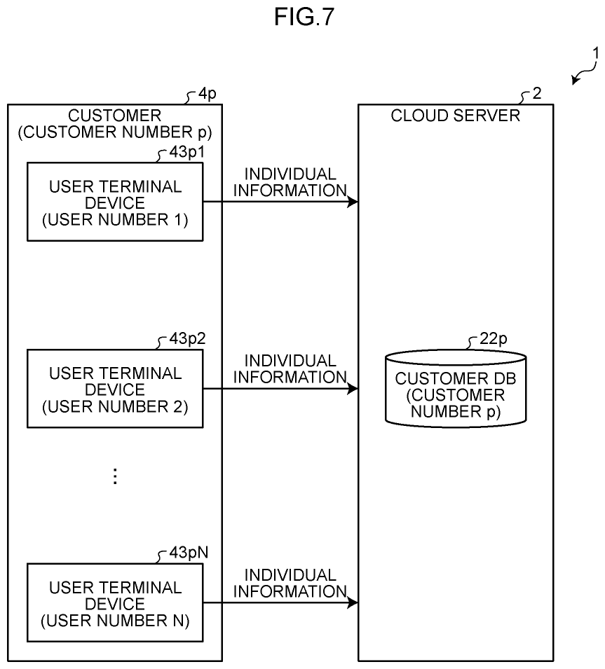

FIG. 7 is a conceptual diagram illustrating an example of a procedure for managing the user information table. In the present disclosure, the user in each of the customers 4p logs into the password-protected individual information management application to register and update the individual information for the user.

Specifically, the individual information for the user transmitted from the user terminal device 43p1 of the customer 4p is stored in the user information table in the customer DB 22p on the cloud server 2.

The individual information for the user transmitted from the user terminal device 43p2 of the customer 4p is stored in the user information table in the customer DB 22p on the cloud server 2.

The individual information for the user transmitted from the user terminal device 43pN of the customer 4p is stored in the user information table in the customer DB 22p on the cloud server 2.

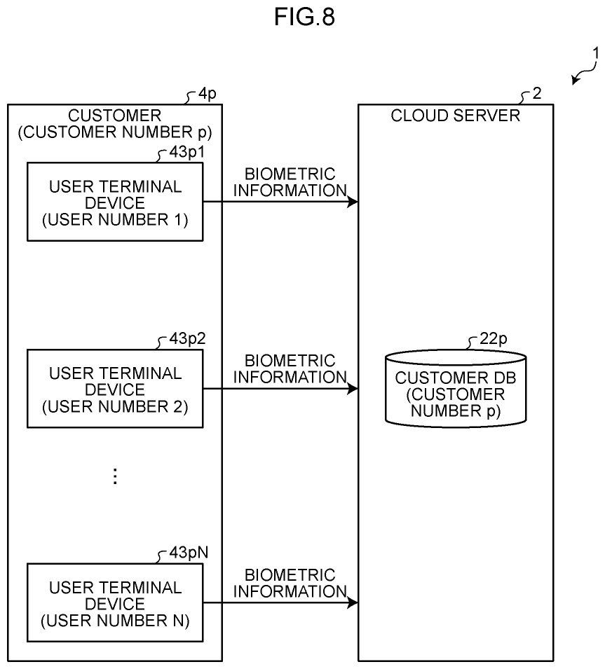

FIG. 8 is a conceptual diagram illustrating an example of a procedure for updating the biometric information table. In the present disclosure, the user in each of the customers 4p logs into the password-protected biometric information acquisition application and uploads the biometric information of the user acquired by the biometric information acquisition device. The biometric information of the user may be acquired at a time preset by the biometric information acquisition application, or at a time specified by the user as needed. The biometric information table is updated based on the uploaded biometric information of the user.

Specifically, the biometric information of the user uploaded by the user terminal device 43p1 of the customer 4p is stored in the biometric information table in the customer DB 22p on the cloud server 2.

The biometric information of the user uploaded by the user terminal device 43p2 of the customer 4p is stored in the biometric information table in the customer DB 22p on the cloud server 2.

The biometric information of the user uploaded by the user terminal device 43pN of the customer 4p is stored in the biometric information table in the customer DB 22p on the cloud server 2.

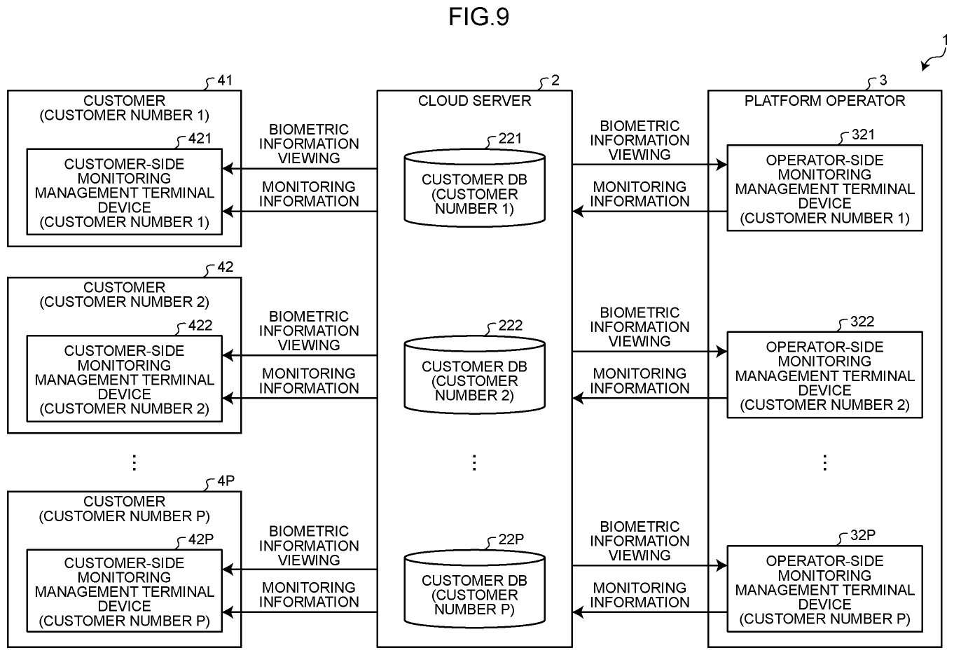

FIG. 9 is a conceptual diagram illustrating an example of a procedure for managing the biometric information table. In the present disclosure, as described above, by using the operator-side monitoring management terminal device 32p, the operator-side monitoring management staff of the platform operator 3 can view the biometric information table of the customer 4p and add thereto the information on the health management for the user as the monitoring information.

As described above, by using the customer-side monitoring management terminal device 42p, the customer-side customer information management staff of each of the customers 4p can view the biometric information table of the customer 4p and refer to the monitoring information added by the operator-side monitoring management staff as the information on the health management for the users. Thus, the qualitative improvement in the health management for the users can be expected.

Specifically, the operator-side monitoring management staff assigned to the customer 41 uses an operator-side monitoring management terminal device 321 to view the biometric information table of the customer 41 stored in the customer DB 221 on the cloud server 2 and add thereto the information on the health management for the user as the monitoring information.

The customer-side customer information management staff of the customer 41 uses a customer-side monitoring management terminal device 421 to view the biometric information table of the customer 41 and refer to the monitoring information added by the operator-side monitoring management staff as the information on the health management for the user. Thus, the qualitative improvement in the health management for the user in the customer 41 can be expected.

The operator-side monitoring management staff assigned to the customer 42 uses an operator-side monitoring management terminal device 322 to view the biometric information table of the customer 42 stored in the customer DB 222 on the cloud server 2 and add thereto the information on the health management for the user as the monitoring information.

The customer-side customer information management staff of the customer 42 uses a customer-side monitoring management terminal device 422 to view the biometric information table of the customer 42 and refer to the monitoring information added by the operator-side monitoring management staff as the information on the health management for the user. Thus, the qualitative improvement in the health management for the user in the customer 42 can be expected.

The operator-side monitoring management staff assigned to the customer 4P uses an operator-side monitoring management terminal device 32P to view the biometric information table of the customer 4P stored in the customer DB 22P on the cloud server 2 and add thereto the information on the health management for the user as the monitoring information.

The customer-side customer information management staff of the customer 4P uses a customer-side monitoring management terminal device 42P to view the biometric information table of the customer 4P and refer to the monitoring information added by the operator-side monitoring management staff as the information on the health management for the user. Thus, the qualitative improvement in the health management for the users in the customer 4P can be expected.

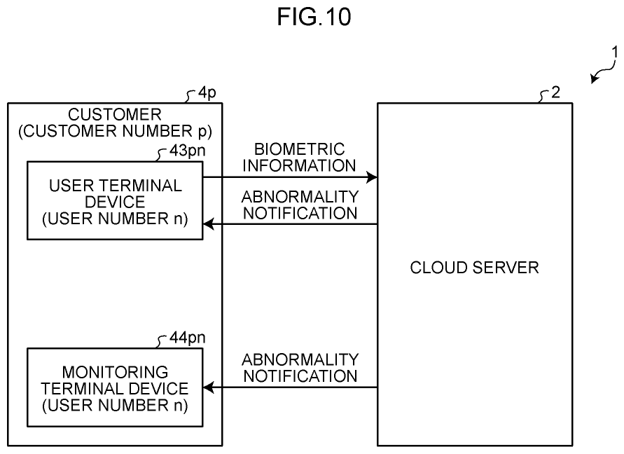

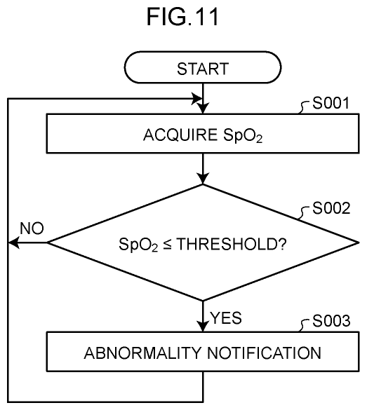

FIG. 10 is a conceptual diagram illustrating an example of a procedure for transmitting the user abnormality notification. FIG. 11 is a flowchart illustrating an example of a process for determination of a biometric information abnormality by the cloud server. FIG. 10 illustrates an aspect of transmission of the user abnormality notification to the user terminal device 43pn and the monitoring terminal device 44pn of the customer 4p. FIG. 11 illustrates an example of acquiring a blood oxygen saturation level (SpO₂) of the user as the biometric information of the user and performing a threshold check on the blood oxygen saturation level.

The cloud server 2 acquires the blood oxygen saturation level (SpO₂) uploaded by the user terminal device 43pn (Step S001), and determines whether the blood oxygen saturation level is equal to or less than a threshold (or below the threshold) (Step S002).

If the blood oxygen saturation level (SpO₂) uploaded by the user terminal device 43pn is greater than the threshold (or equal to or greater than the threshold) (No at Step S002), Steps S001 and S002 are repeated.

If the blood oxygen saturation level (SpO₂) uploaded by the user terminal device 43pn is equal to or less than the threshold (or below the threshold) (Yes at Step S002), the abnormality notification indicating that the blood oxygen saturation level (SpO₂) of the user has been an abnormal value is transmitted to the user terminal device 43pn and the monitoring terminal device 44pn of the customer 4p (Step S003).

This operation enables a quick response when an abnormality in health conditions of the user has occurred.

As described above, in the health management system 1 according to the embodiment, the cloud server 2 includes the master DB 21 and the customer DBs 22p. The master DB 21 stores therein the customer numbers (customer IDs) of the customers 4p, and the customer DBs 22p correspond to the respective customers 4p and each store therein the individual information and the biometric information of each of the users of the corresponding customer 4p.

In the health management system 1 according to the embodiment, the customer DBs 22p provided corresponding to the customers 4p are associated with the customer numbers (customer IDs) stored in the master DB 21. This configuration can bring efficiency to the management of the customer information for each of the customers 4p, the individual information for the users, and the biometric information of each of the users on the cloud server 2.

As described above, in the health management system 1 according to the embodiment, each of the multiple operator-side monitoring management terminal devices 32p corresponding to the customers 4p has authority to access the customer DB 22p storing therein the biometric information table of the corresponding customer 4p. Thus, the security among the customers 4p is ensured.

As described above, in the health management system 1 according to the embodiment, by using the operator-side monitoring management terminal device 32p, the operator-side monitoring management staff can view the biometric information table of the customer 4p and add thereto the information on the health management for the user as the monitoring information. By using the customer-side monitoring management terminal device 42p, the customer-side monitoring management staff can view the biometric information table of the customer 4p and refer to the monitoring information added by the operator-side monitoring management staff as the information on the health management for the users. Thus, the qualitative improvement in the health management for the users can be expected.

Furthermore, as described above, in the health management system 1 according to the embodiment, the cloud server 2 determines the health conditions of the user based on the biometric information. If the occurrence of the abnormality in health conditions of the user is detected, the cloud server 2 transmits, to the user terminal device 43pn, the abnormality notification indicating that the abnormality in health conditions of the user has occurred, and also transmits the abnormality notification to the monitor terminal device (monitoring terminal device 44p) owned by the monitor d by the user. This operation enables a quick response when the abnormality in health conditions of the user has occurred.

While the preferred embodiment of the present disclosure has been described above, the present disclosure is not limited to the embodiment described above. The content disclosed in the embodiment is merely an example, and can be variously modified within the scope not departing from the gist of the present disclosure. For example, any modifications appropriately made within the scope not departing from the gist of the present disclosure also naturally belong to the technical scope of the present invention.

What is claimed is:

1. A health management system comprising:
a server;
a user terminal device through which a user in a customer uploads biometric information to the server;
an operator-side monitoring management terminal device through which the biometric information uploaded to the server is viewable and monitoring information on health management for the user is addable to the biometric information; and
a customer-side monitoring management terminal device through which the biometric information uploaded to the server and the monitoring information added to the biometric information are viewable.

2. The health management system according to claim 1, wherein
the server comprises a plurality of customer databases corresponding to a plurality of the customers, and
each of the customer databases is configured to store the biometric information of a plurality of the users in a corresponding one of the customers.

3. The health management system according to claim 2, wherein
the server comprises a master database storing customer identifiers (IDs) of the customers, and
each of the customer databases is associated with a corresponding one of the customer IDs of the customers.

4. The health management system according to claim 3, comprising:
a plurality of the operator-side monitoring management terminal devices corresponding to the customers; and
a plurality of the customer-side monitoring management terminal devices owned by the respective customers, wherein
each of the operator-side monitoring management terminal devices has authority to access the customer database of a corresponding one of the customers, and
each of the customer-side monitoring management terminal devices has authority to access the customer database of the customer owning the customer-side monitoring management terminal device.

5. The health management system according to claim 4, comprising a plurality of the user terminal devices owned by the respective users, wherein
the server is configured to transmit, if detecting an occurrence of abnormality in health conditions of one of the users based on the biometric information, an abnormality notification to the user terminal device owned by the user with the detected abnormality in health conditions.

6. The health management system according to claim 5, comprising a plurality of monitor terminal devices owned by monitors designated by the users, wherein
each of the customer databases is configured to store contact information for each of the monitor terminal devices of a corresponding one of the customers.

7. The health management system according to claim 6, wherein each of the customer databases is associated with a corresponding one of the customer IDs of the customers.

8. The health management system according to claim 7, wherein the server is configured to transmit, if a numerical value indicating the biometric information of the user is an abnormal value, the abnormality notification to the monitor terminal device owned by the monitor designated by the user.

9. The health management system according to claim 1, wherein the user terminal device comprises a biometric information acquisition device configured to acquire the biometric information of the user.

10. The health management system according to claim 9, wherein the biometric information acquisition device is a smart ring.

11. The health management system according to claim 10, wherein the biometric information acquisition device comprises an optical sensor.

12. The health management system according to claim 11, wherein the optical sensor is an organic photodiode (OPD).
